(19) United States
(12) Patent Application Publication
(10) Pub. No.: US 2018/0214504 A1
(43) Pub. Date: Aug. 2, 2018

(54) HEALTH SUPPLEMENT FOOD FOR PROMOTION OF HAIR GROWTH, MITIGATION AND ALLEVIATION OF HAIR LOSS, REGULATION OF BLOOD GLUCOSE, AND ALLEVIATION AND RELIEF OF GASTRIC DISORDER SYMPTOMS

(71) Applicant: Kyung Il KIM, Seoul (KR)

(72) Inventor: Kyung Il KIM, Seoul (KR)

(21) Appl. No.: 15/558,183

(22) PCT Filed: Mar. 11, 2016
(86) PCT No.: PCT/KR2016/002450

§ 371 Date: Sep. 13, 2017

(30) Foreign Application Priority Data
Apr. 8, 2015 (KR) 10-2015-0049688

(51) Int. Cl. A61K 36/8994; A23L 33/105; A23L 7/10; A23L 11/00; A23L 19/00; A23L 17/60; A61K 36/48; A61K 36/185; A61K 36/899; A61K 36/605; A61K 36/70; A61K 36/288; A61K 36/484; A61K 36/03; A61K 36/33; A61K 9/20; A61P 17/14; A61P 3/08; A61P 1/00

(52) U.S. Cl. CPC A61K 36/8994; A23V 2002/00; A23L 7/198; A23L 11/05; A23L 19/01; A23L 17/60; A61K 36/48; A61K 36/185; A61K 36/899; A61K 36/605; A61K 36/70; A61K 36/288; A61K 36/484; A61K 36/03; A61K 36/33; A61K 9/20; A61P 17/14; A61P 3/08; A61P 1/00; A23L 33/105

(57) ABSTRACT

The present invention relates to a health supplement food. More particularly, the present invention relates to a health supplement food prepared by mixing dried powders, wherein the dried powders include 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to % by weight of dandelion, 1 to 10% by weight of licorice, 2 to 15% by weight of kelp, and 5 to 15% by weight of cactus based on solid content. The health supplement food may exhibit effects such as promotion of hair growth, alleviation and mitigation of hair loss, regulation of blood glucose, or alleviation and relief of gastric disorder symptoms.

### TECHNICAL FIELD

The present invention relates to a health supplement food for promotion of hair growth, alleviation and mitigation of hair loss, regulation of blood glucose, and alleviation and relief of gastric disorder symptoms.

### BACKGROUND ART

Health supplement foods are food groups that are produced by concentrating, refining, and mixing specific foods or special ingredients that are beneficial to the body. These health supplement foods can be considered special-purpose foods that are consumed by consumers for the purpose of promoting health.

In general, health supplement foods refer to foods that are expected to be effective for maintenance and enhancement of health due to special ingredients thereof when compared with ordinary food and are considered medically effective in maintaining human health. Such health supplement foods are consumed in hopes of preventing or treating diseases and are widely commercialized.

As interest in health has greatly increased in recent years, consumer interest in health supplement foods and nutritional supplements has also greatly increased.

If all nutrients can be obtained from food, there is no thing better than that. However, modern people have difficulty in evenly consuming enough nutrients due to irregular eating habits. Accordingly, people who do not have healthy eating habits or people who cannot afford healthy meals can sufficiently obtain essential nutrients from health supplement foods to supplement deficient nutrients.

Health supplement foods contain many ingredients. Accordingly, when many products are taken at the same time, each of the ingredients therein may interfere with one another's absorption or cause chemical reactions in the body and the like, which may lead to unpredictable results. Therefore, it is important to select health supplement foods that are necessary for each person's body. Since even good health supplement foods may cause toxicity in people with certain diseases, it is necessary to check which diseases a consumer has before taking a health supplement food.

Meanwhile, various health supplement foods that are useful for various diseases are commercially available, and patents thereon are also being applied continuously.

Korean Patent No. 10-1094157 proposes a health supplement food for alleviating diabetes mellitus prepared using medicinal herbs. Here, the composition of the medicinal herbs may include ginseng, Lycii fructus, Lycii radicis Cortex, Atractylodis rhizoma Alba, Polygonum multiflorum Thunberg, Dioscoreae rhizoma, Rehmanniae radix, Corni fructus, Acanthopanacis senticosi radix et rhizoma, Momordica charantia, Trichosanthis radix, Prunellae spica, Syzygii flos, Foeniculum vulgare Miller, Cinnamomi cortex, Cuscutae semen, Cnidium monnieri (L.) Cusson, Loranthi ramulus et folium, Hoveniae semen seu fructus, Poria cocos, Batryticatus bombyx, Cordyceps, yeast, and the like.

In addition, Korean Patent No. 10-1445573 proposes an enzyme solution prepared in a short period by pulverizing Cudrania tricuspidata, Momordica charantia, Helianthus tuberosus L., Ulmi cortex, and Artemisia annua Linné and fermenting the same using Effective Microorganisms (EM) and Cudrania tricuspidata as a main raw material. This prepared enzyme solution is disclosed to be suitable for easy intestinal toxin release, easy food digestion and absorption, dieting, and diabetes mellitus alleviation.

Korean Patent No. 10-0578665 proposes a functional health food for alleviating gastrointestinal disease symptoms containing an herbal composition mixture prepared by mixing herbal extracts, such as Poria sclerotium cum pini radix, Cinnamomi ramulus, and Phellinus linteus, with Pycnogenol.

In addition, Korean Patent No. 10-0982044 discloses a health supplement food containing black soy beans, black sesame seeds, Chinese yam, Atractylodis rhizoma Alba, safflower seeds, pine needles (red pine needles), and additives such as general raw rice wine (rice wine), clay soil powder, and ginger. Here, the health supplement food has prevention effects on gout, heart disease, liver disease, poor blood circulation, kidney disease, and the like when taken for a long time.

As such, there are health supplement foods with specific compositions which are used as adjunct treatments for various diseases. A variety of health supplement foods for improving hair growth or alleviating hair loss as well as the aforementioned diseases has been proposed.

Hair loss usually occurs due to genetics. However, unlike the past, hair loss caused by eating habits, lifestyle, and stress is rapidly increasing and is occurring among all ages of men and women.

Korean Patent Publication Application No. 2004-0006704 proposes a health supplement food exhibiting effects on hair loss prevention and hair growth effect. In particular, the health supplement food is prepared by mixing more than eight kinds of medicinal herb extracts, organic germanium (Ge-132), and refined deep water (diluted deep water).

In addition, Korean Patent No. 10-0733841 proposes a health supplement food for anti-aging or preventing hair loss and facilitating hair growth which is prepared by drying and pulverizing 40 to 50% by weight of black beans, 20% by weight of black sesames, 10% by weight of Polygonum multiflorum Thunberg, and 10% by weight of kelp with 10 to % by weight of one or more selected from the group consisting of pine needles, Lycii fructus, green tea, walnuts, and pine nuts and processing the same in an immediately eatable form, in a mixed powder form, or in a pill shape.

In addition, Korean Patent No. 10-1039439 refers to a functional health food for preventing hair loss and promoting hair growth which is prepared by roasting black sesames, black beans, and black rice and then processing the same into a pill. When this functional health food is taken, there are no side effects, organs helping digestion and absorption are activated and become healthy, and hair loss prevention and hair growth promotion can be accomplished.

In addition, Korean Patent No. 10-1244302 proposes a health supplement food prepared using medical herbs such as ginseng, Polygonum multiflorum Thunberg, Angelicae gigantis radix, Cnidium officinale Makino, Astragalus membranaceus Bunge, Atractylodis rhizoma Alba, Crataegi fructus, Rehmanniae radix preparata, Schisandra chinensis Baillon, Dioscoreae rhizoma, and honey. The health supplement food is disclosed as preventing aging by enhancing kidney and liver functions, and thus preventing hair loss and promoting hair growth.

In addition to the aforementioned patent applications, a variety of new products for promotion of hair growth and prevention of hair loss are emerging every year. When these products are released, many people buy the same. However, since hair loss prevention effects thereof are not apparent, people again buy newly released products. Such a phenomenon is repeated, whereby people waste money and time.

### RELATED ART DOCUMENT

### Patent Documents

(Patent Document 1) Korean Patent No. 10-1094157

(Patent Document 2) Korean Patent No. 10-1445573

(Patent Document 3) Korean Patent No. 10-0982044

(Patent Document 4) Korean Patent Publication Application No. 2004-0006704

(Patent Document 5) Korean Patent No. 10-0733841

(Patent Document 6) Korean Patent No. 10-1039439

(Patent Document 7) Korean Patent No. 10-1244302

### DISCLOSURE

### Technical Problem

Accordingly, the present applicant performed various researches into a health supplement food that exhibits a variety of efficacies without side effects even when taken for a short time, and thus, alleviates hair loss, diabetes mellitus, and gastrointestinal disease symptoms. The present applicant selected various cereals, such as black beans, and medicinal plants such as mulberry leaves, mulberry fruits, and Polygonum multiflorum Thunberg, and mixed the same in a specific content ratio. As a result, the present application confirmed that, when various cereals and medicinal plants are mixed in a specific content ratio, hair loss may be prevented, hair growth may be promoted, and gastrointestinal disease may be alleviated by aiding regulation of blood glucose.

Therefore, the present invention has been made in view of the above problems, and it is one object of the present invention to provide a health supplement food enabling promotion of hair growth, alleviation and mitigation of hair loss, regulation of blood glucose, or alleviation of gastrointestinal disorder symptoms.

### Technical Solution

In accordance with one aspect of the present invention, provided is a health supplement food, wherein the health supplement food is prepared by mixing dried powders, wherein the dried powders include 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to % by weight of dandelion, 1 to 10% by weight of licorice, 2 to 15% by weight of kelp, and 5 to 15% by weight of cactus based on solid content.

Here, the mulberry leaves and the mulberry fruits may be simultaneously harvested from Morus bombycis, wherein the mulberry fruits are harvested when blue, red, and dark purple mulberry fruits are mixed.

The health supplement food may have a pill, granule, tablet, powder, capsule, or beverage formulation.

### Advantageous Effects

As apparent from the fore-going, the present invention advantageously provides a health supplement food including corresponding ingredients in an optimal ratio, thereby exhibiting effective and safe effects on promotion of hair growth, alleviation and mitigation of hair loss, regulation of blood glucose, or alleviation and relief of gastric disorder symptoms.

Such a health supplement food has no side effect and exhibits specific effects regardless of gender, age, and constitution even when taken for a short time, thereby increasing consumer satisfaction.

### BEST MODE

The present invention proposes a functional health supplement food having a variety of effects.

In the present specification, a health supplement food refers to a food prepared or processed by isolating, extracting, concentrating, refining, or mixing ingredients per se, as raw materials, in food materials or specific ingredients therein and taken to obtain efficacy in the physical and physiological effects on the body.

In such a health supplement food, the contents of a variety of grain materials and medicinal plants are limited in a specific range, thereby maximizing the efficacy of each of the materials.

In particular, the health supplement food is prepared by mixing dried powders, wherein the dried powders include 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to 15% by weight of dandelion, 1 to 10% by weight of licorice, 2 to 15% by weight of kelp, and to 15% by weight of cactus based on solid content. Within these content ranges, the efficacy of each of the materials may be maximized. When the contents of the materials are outside the range, effects of the present invention might not be secured. Accordingly, each of the materials is suitably used within the ranges.

Hereinafter, the composition is described in more detail.

First, the health supplement food according to the present invention includes black beans and black sesame seeds as representative black foods.

Black food refers to foods with a black surface. Proteins in black beans and black sesame seeds, as black foods, are effective in preventing hair loss. In addition, since a collagen-activating ingredient is contained in black beans and black sesame seeds, these foods enhance skin elasticity. Further, since these foods are rich in selenium, the foods are known to be good for diseases related to selenium deficiency.

In particular, black beans, which are called Glycine semen nigra, collectively refer to black Fabaceae. As examples of black beans, there are Glycine max (L.) MERRILL (Rhynchosia volubilis, Momordica charantia, black soy beans, or Glycine max), and the like. Thereamong, Glycine max (L.) MERRILL is preferred. As needed, a mixture of two or more kinds of Glycine max (L.) MERRIL may be used. In Ben Cao Gang Mu, black beans are disclosed as controlling the kidneys, eliminating edema, stimulating blood circulation, and releasing poisons from the body. Also, in Korean medicine, black beans are considered to “rapidly restore destroyed human tissues due to excellent detoxifying ability thereof.” Black beans contain not only high-quality proteins but also nutrients such as lipids, vitamin B1, vitamin B2 and vitamin E, compared to ordinary soybeans. In addition, black beans contain cysteine, an essential ingredient for hair growth, thereby being effective in preventing hair loss. In addition, cyanidin-3-glucoside, as a pigment in black soybean, is one of the most broadly researched anthocyanins and has various effects such as an anti-cancer effect, a gastrointestinal protection effect, an inflammation inhibition effect, an antioxidant effect, and an ulcer prevention effect. In particular, when anthocyanins pass through the gastrointestinal tract in the body, sugars are separated from molecular structures of anthocyanins, thereby being converted into anthocyanidins. This converted structure is known to have stronger physiological activity in the human body.

Black sesame, scientific name Sesamum indicum L., is especially good for prevention of adult diseases and prevention of aging due to excellent antioxidant effects. In addition, black sesame is known to exhibit various effects by aiding kidney and brain functions. When the hair and skin are not glossy and the skin is dry, the hair and skin may become glossy by eating black sesame seeds. In addition, lipid ingredients in black sesame seeds affect scalp health, thereby being effective in preventing hair loss. Further, black sesame contains an antioxidant ingredient called gamma-tocopherol (a form of vitamin E).

Along with the aforementioned black foods, the health supplement food according to the present invention includes corn (Zea mays) as a representative yellow food. Yellow foods are rich in antioxidants suppressing aging. Thereamong, corn enhances immunity due to being rich in beta-carotene and vitamin B group and has antioxidant effects. In addition, corn aids hair growth and has a gastrointestinal disease relief effect.

Adlay (Coix lachryma-jobi var. mayuen (ROMAN STAPF)) is an annual herbaceous plant belonging to family Gramineae. In Donguibogam, adlay is disclosed to be effective in blackening hair and preventing hair loss. In oriental medicine, adlay is called Coicis Semen and is used as a medicine. Adlay is known to help alleviate hair loss and strengthen muscles, protect the spleen and stomach, promote insulin secretion in the body, and alleviate diabetes mellitus by lowering blood glucose. In the past, adlay was eaten as a meal and was usually eaten in porridge form. Recently, effects of adlay on adult diseases and tonic effects thereof have been noted, whereby processed adlay foods, such as adlay tea, are being used.

Sorghum (Sorghum bicolor MOENCH), as an annual plant belonging to family Gramineae, is written as [symbol] or [symbol] in Chinese. Since sorghum is rich in carbohydrates, fats, proteins, vitamins, and mineral fiber enzymes and contains a large amount of copper and zinc, sorghum is known to suppress activation of enzymes related to hair loss and facilitate hair growth. In addition, sorghum is known to aid digestion, to stop diarrhea and to stabilize the mind. Further, sorghum has prevention effects on vascular diseases and various adult diseases by removing cholesterol from the body to improve blood circulation, strengthening blood vessels, and clearing the blood.

The health supplement food according to the present invention includes a variety of medicinal plants along with the aforementioned cereals.

In particular, to improve various efficacies and alleviate various diseases referred to in the present invention, mulberry leaves and mulberry fruits obtained from mulberry and Polygonum multiflorum Thunberg are used together.

Mulberry was originally cultivated to farm silkworms. Most parts, such as the root bark, fruits, and leaves, of mulberry are used as medicine. Thereamong, in the present invention, mulberry leaves and mulberry fruits are used.

Throughout the world, there are about 10 species of mulberry. Morus bombycis, Morus bombycis for. Dissecta, Morus mongolica, Morus tiliaefolia Makino, and Cudrania tricuspidata grow in Korea. Mulberry prevents diabetes mellitus and has excellent effect in lowering the blood glucose of diabetics. Preferably, in the present invention, leaves and fruits obtained from Morus bombycis are used. As needed, Morus bombycis for. Dissecta, Morus mongolica, Morus tiliaefolia Makino, and Cudrania tricuspidata may be used together.

Mulberry leaves are written as [symbol] in Chinese. Efficacies of mulberry leaves are documented in Chinese and Korean traditional medical books and such mulberry leaves have been used as medicine for generations. In mulberry leaves, a large amount of chlorophyll regulating blood glucose and maintaining the homeostasis of the body is contained. In China's “Ben Cao Gang Mu,” “roots, leaves, and fruits of mulberry are all used as medicine” is disclosed. In addition, in China's “Ben Cao Gang Mu,” 18 products from mulberry, such as mulberry leaves and mulberry fruits, are effective against 177 diseases such as beriberi, diabetes mellitus, and snake bites or worms.

Mulberry fruit, which is called Morus bombycis Koidz), is a sap fruit similar to strawberries. Mulberry fruit is initially blue, but gradually reddens. When ripe, the color of a mulberry fruit changes from purple to dark purple. In the decoction section of “Donguibogam,” descriptions of a mulberry fruit are as follows: “black mulberry fruit, which is a gathering of the spirit of a mulberry tree, alleviates diabetes mellitus, has beneficial effects on five organs, and, when a lot of mulberry fruit is taken, hunger is alleviated,” “mulberry fruit elevates mood and enhances hearing” and “when mulberry fruit is taken for a long time, white hair turns black and aging is prevented.” Mulberry fruit is sweet and the property thereof is cold. Such a mulberry fruit turns hair black and entirely enhances all functions of the body. Also, in old books and folk remedies, mulberry fruit is known to have anti-diabetic efficacy.

In particular, in the present invention, a mixture of blue, red, and dark purple mulberry fruits is harvested from Morus bombycis and/or Cudrania tricuspidata. In this case, mulberry leaves are also harvested. When collection is performed in this manner, the effects of the present invention may be maximized.

Along with the mulberry leaves and mulberry fruits, Polygonum multiflorum Thunberg is used. Roots, which extend to the ground, of Polygonum multiflorum Thunberg have a round tuberous root shape. In the present invention, tuberous roots of Polygonum multiflorum Thunberg (polygonaceae), as a climbing perennial herbaceous plant, are used. In herbal medicine, Polygonum multiflorum Thunberg is known to boost energy, strengthen muscles and bones, add stamina, darken hair, make the face beautiful, and alleviate postpartum depression and leucorrhea. In “Donguibogam,” Polygonum multiflorum Thunberg has an effect on circulatory system diseases, all kinds of congestion, and restoration and, when taken for a long time, white hair becomes black. In addition, Polygonum multiflorum Thunberg is known to strengthen the functions of the liver and kidneys and strengthen the muscles and bones.

Dandelion (Taraxacum mongolicum Hand.-Mazz.) is a perennial plant belonging to family Asteraceae. Taraxacum platycarpum Dahlst. (Taraxacum mongolicum Hand.-Mazz), Taraxacum coreanum Nakai (Taraxacum albidum Dahist), Taraxacum coreanum Nakai var. flavescens Kitamura, Taraxacum manshuricum Nakai et Koidzumi (Taraxacum ohwianum Kitamura), Taraxacum hallaisanense Nakai, Taraxacum officinale Weber et Wiggers (Leontodon taraxacum L.), and Taraxacum erythrospermum Andrz. are found in Korea. According to the “Youngnamchaeyakjeon,” dandelion is disclosed as treating stomach pain, abdominal pain, and the like when it is dried over a fire such that the efficacy thereof remains weakly and is taken with alcohol. In addition, since dandelion acts to strengthen the stomach and intestines, it is used as an herbal medicine for treating nausea, indigestion, anorexia, diarrhea, constipation, and the like. In particular, although a dandelion used in the present invention is not specifically limited, dandelion may be used with roots thereof. The effects desired in the present invention may be sufficiently secured only when dandelion is used together with roots thereof.

Licorice (Glycyrrhiza uralensis Fischer) is a perennial herbaceous plant belonging to family Leguminosae. Roots of licorice are dried and used as a medicine. Licorice was named after sweat taste thereof. Licorice neutralizes the toxicity of all medicines, alleviates coughs and phlegm, and neutralizes all medicines. In Donguibogam, licorice is disclosed as regulating heat and frailty of five organs and six hollow viscera, normalizing the eye, nose, mouth, ear, and natural functions such as defecation, making all blood vessels communicate, strengthening the muscles and bones, and improving nutritional state. In addition, in accordance with recent research, licorice was reported to have ulcer inhibitory action of inhibiting gastric acid secretion by promoting interferon release from the body and protecting the gastric mucosa. In addition, it was reported that, when licorice is taken in the case of gastroduodenal ulcer, it has significant pain alleviation effects, such as pain removal or relief.

Kelp (Saccharina japonica), which is a seaweed belonging to family Laminariaceae, is also written as [symbol] or [symbol] in Chinese. Since kelp contains dietary fiber called alginic acid, it is effective in reducing cholesterol levels and blood pressure. In addition, kelp lowers blood glucose by delaying penetration of glucose into the blood and aiding digestion and absorption of sugars. Since kelp contains carbohydrates, such as mannitol and laminarin produced by carbon assimilation, proteins, iodine, iron, calcium, sulfur, vitamin B2, and amino acids such as glutamic acid, as well as various pigments such as carotene, xanthophyll and chlorophyll, it is good for hair loss prevention. In addition, since kelp contains an oxo ingredient as a hair growth promoter, hair loss may be prevented and hair becomes glossy when kelp is taken continuously.

There are more than 10,000 cactus species throughout the world. Opuntia is native to Korea. As Opuntia, Opuntia ficus-indica or Opuntia Humifusa may be used. In general, cactus native to Jeju Island, etc. is called Opuntia ficus-indica, and cactus capable of surviving the winter season in the interior is called Opuntia Humifusa. Preferably, Opuntia Humifusa is used. As needed, only Opuntia ficus-indica or a mixture of Opuntia ficus-indica and Opuntia Humifusa may be used. Both purple fruits (nopal) and stems (nopalitos) of cactus are edible and have effect on improvement of hair growth and alleviation of gastrointestinal diseases such as gastroenteritis, gastritis and gastric ulcer.

Here, cactus is preferably used in a health supplement food for regulation of blood glucose or alleviation of gastrointestinal diseases as well as promotion of hair growth and alleviation of hair loss. Here, the content of the cactus is 5 to 10% by weight based on the total weight of a composition. When the content of the cactus is outside this range, hair growth promotion effect, hair loss alleviation effect, blood glucose regulation effect, or gastrointestinal disease alleviation effect might not be sufficiently secured.

Additionally, along with the aforementioned black beans, black sesame seeds, corn, adlay, sorghum, mulberry leaves, mulberry fruits, Polygonum multiflorum Thunberg, dandelion, licorice, and kelp, various materials capable of alleviating each disease may be further included.

The health supplement food including the aforementioned materials may be prepared in various forms.

As mentioned above, the health supplement food of the present invention refers to a normal food and includes both food and drink. Accordingly, the health supplement food of the present invention is not limited to a specific formulation. For example, the health supplement food may be prepared in various formulations such as a pill, granule, tablet, powder, capsule, or beverage formulation. These formulations are easy to carry and take anywhere, anytime.

A method of preparing the health supplement food of the present invention is not specifically limited, and may be any publicly known method.

Preferably, the health supplement food is prepared in a powder formulation. In this case, the health supplement food according to the present invention may be prepared according to the following step:

S1) a step of washing and drying each of materials;

S2) a step of pulverizing the dried materials; and

S3) s step of mixing the pulverized materials in a predetermined content ratio to prepare a powder mixture.

Such a powder mixture may be directly taken or may be dissolved in a beverage such as water, honey, or milk.

In addition, to produce the health supplement food in a pill formulation, water is added to the powder mixture and then kneading is performed, followed by preparing the same in a pill formulation by means of a pill manufacturing machine. The prepared pills are sterilized and dried in an electric hot air sterilization drier for 24 hours, followed by vacuum packaging.

The water may be contained in an amount which is sufficient to knead the mixed powder and prepare the same in a pill formulation. For example, the water may be included in a weight ratio of 1:0.05 to 0.5.

As needed, a mixture of water and a fermented mulberry fruit solution may be used instead of water, or wheat flour, flour paste, starch syrup or honey may be used to facilitate kneading. The fermented mulberry fruit solution is prepared by fermenting with sugar. A preparation method thereof is not specifically limited in the present invention.

The content of the fermented mulberry fruit solution, the wheat flour, the flour paste, the starch syrup, or the honey is not specifically limited in the present invention, and may be a content within which a pill formulation may be easily prepared and the same of the prepared pill may be maintained for a certain period.

Of course, using a granule manufacturing machine instead of the pill manufacturing machine, the health supplement food may be molded in a granular shape having a diameter of 0.5 to 1.5 mm. In addition, the prepared granules may be extrusion-molded in a proper amount, thereby being prepared in a tablet or powder formulation, or in a capsule formulation by filling capsules with the same.

Additionally, various additives used in a health supplement food may be added to the powder mixture.

As the food additives, for example, a carbonation agent, sugars, such as monosaccharides, disaccharides, polysaccharides, and sugar alcohols, flavors, such as thaumatin, stevia extract, saccharin, and aspartame, nutrients, vitamins, edible electrolytes, flavors, colorants, pectic acid, alginic acid, organic acids, protective colloid thickeners, a pH adjuster, a stabilizer, a preservative, glycerin, a carbonation agent, and the like may be used. These food additives may be used in an amount of 5% by weight based on a total amount of a resultant composition.

The various formulations of the health supplement food prepared according to the aforementioned method may be individually packaged in a suitable single dose or packaged in containers. The single dose may be varied depending upon a formulation, but, in the case of the pill formulation as in the first and second embodiments, 15 to 30 tablets, preferably 20 tablets, may be taken three times a day. In the case of the powder formulation as in the third embodiment, 1 to 3 tablespoons, preferably 2 tablespoons, may be taken three times a day. Here, an administration method may be varied depending upon a formulation of the health supplement food, and may be changed depending upon the condition and weight of a user and the state, type, and duration of a disease.

The health supplement food according to the present invention has effects on promotion of hair growth, alleviation and mitigation of hair loss, regulation of blood glucose, or alleviation of gastrointestinal disorder symptoms.

First, the health supplement food according to the present invention has effects on promotion of hair growth, and alleviation and mitigation of hair loss.

Hair grows for about 3 to 6 years, falls off, and has a rest period for about 2 to 3 months, and this hair growth cycle is repeated again. In a normal case, about 50 to 70 hairs are lost per day, and about 2 to 5% of all hair follicles are resting at any given time. A symptom wherein hair follicles in a resting state or hair follicles from which hair does not grow at all are increasing is called hair loss.

Main causes of hair loss in men are heredity and male hormones, and a main cause thereof in women is female hormones. In addition to hormone-induced hair loss, hair loss may occur due to malnutrition, drug abuse, childbirth, fever, and surgery. Temporary hair loss occurs when stress is severe. In addition, seborrheic dermatitis, dandruff, poor hair care, and the like may cause hair loss.

With regard to such hair loss, the health supplement food according to the present invention may be taken.

In accordance with a preferred first embodiment of the present invention, a method of preparing a pill formulation for promotion of hair growth and alleviation and mitigation of hair loss may include the following steps:

S1) a step of cleanly washing black beans, black sesame seeds, corn, adlay, and sorghum, mulberry leaves, mulberry fruits, Polygonum multiflorum Thunberg, dandelion, licorice, kelp, and cactus and then drying the same in the sun until water is completely removed therefrom, followed by completely drying in a warm room for two to three days;

S2) a step of pulverizing each of the materials and preparing a powder;

S3) a step of mixing the prepared powders such that 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to 15% by weight of dandelion, 1 to 10% by weight of licorice, 2 to 15% by weight of kelp, and to 15% by weight of cactus are included, thereby preparing a powder mixture;

S4) a step of mixing a fermented mulberry fruit solution and water in a ratio of 1:1;

S5) a step of mixing the powder mixture, and the mixture including the mixed fermented mulberry fruit solution and the water in a weight ratio of 1:0.05 to 0.5 and kneading the same; and

S6) a step of formulating the kneaded mixture of S5 in a pill formulation having a diameter of 0.5 to 0.8 mm, thereby preparing a pill formulation.

It was confirmed that, when such a pill formulation was taken, hair loss was prevented or a hair loss rate was decreased. Further, it was confirmed that hair growth was promoted, and growth of black hair, instead of gray hair, was observed in some clinical trials.

In addition, the health supplement food according to the present invention has effect on blood glucose regulation.

Diabetes mellitus, which refers to a state in which the body does not properly use food and thus the level of glucose (blood sugar) in the blood is much higher than that of normal people, is named due to the characteristic that glucose used as energy in the body is released through urine.

A mechanism related to a cause of diabetes mellitus has yet to be revealed, but diabetes mellitus is known to be caused by genetics, viruses, obesity, aging, dietary habits, stress, medication, and the like. Diabetes mellitus per se may be controlled to a certain extent through management, but it is also important to reduce risk factors for diabetic complications.

Diabetes mellitus may be treated with insulin and oral hypoglycemic agents for controlling blood glucose, aspirin, blood pressure medication, cholesterol regulators, antidepressants, and sleeping pills for preventing complications, and the like. However, these medicines may cause adverse drug effects such as diarrhea, nausea, and headache. In addition, when insulin is injected into the same site every day, absorption of insulin may be poor and tissues may be damaged.

Accordingly, blood glucose may be controlled by taking the health supplement food of the present invention, thereby may increasing the interval of insulin injection or reducing the frequency of insulin injection.

In accordance with a preferred second embodiment of the present invention, a method of preparing a pill formulation for promotion of hair growth and alleviation and mitigation of hair loss may include the following steps:

S1) a step of cleanly washing black beans, black sesame seeds, corn, adlay, and sorghum, mulberry leaves, mulberry fruits, Polygonum multiflorum Thunberg, dandelion, licorice, kelp, and cactus and then drying the same in the sun until water is completely removed therefrom, followed by completely drying in a warm room for two to three days;

S2) a step of pulverizing each of the materials and preparing a powder;

S3) a step of mixing the prepared powders such that 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to 15% by weight of dandelion, 1 to 10% by weight of licorice, 2 to 15% by weight of kelp, and to 15% by weight of cactus are included, thereby preparing a powder mixture;

S4) a step of mixing a fermented mulberry fruit solution and water in a ratio of 1:1;

S5) a step of mixing the powder mixture, and the mixture including the mixed fermented mulberry fruit solution and the water in a weight ratio of 1:0.05 to 0.5 and kneading the same; and

S6) a step of formulating the kneaded mixture of S5 in a pill formulation having a diameter of 0.5 to 0.8 mm, thereby preparing a pill formulation.

It was confirmed that, when the pill-type health supplement food was taken, blood glucose was lowered, as measured by a self-monitoring blood glucose test. Accordingly, it was confirmed that an insulin injection interval might be increased, the dose of the drug might be lowered, and blood glucose might be controlled to some extent without insulin injection.

In addition, the health supplement food according to the present invention has effects on alleviation and relief of gastric disorder symptoms.

Gastritis is a disease causing nausea, abdominal bloating, dyspepsia, heartburn, and the like due to inflammation of the mucous membrane of an inner wall of the stomach. The symptom that an inner wall of the stomach is torn due to progress of gastritis is called stomach ulcer (peptic ulcer). Stomach ulcer (peptic ulcer) may be accompanied by gastric bleeding along with severe pain.

Gastrointestinal diseases such as gastritis and gastric ulcers have a variety of causes. For example, stress, spicy foods, overeating, foods inducing excess gastric acid secretion, such as alcohol, caffeine, and carbonated beverages, infection by Helicobacter pylori, and frequent medication of drugs stimulating the stomach (anti-inflammatory drugs, steroids, an iron agent, a calcium agent, vitamin C, and the like) may cause gastritis. In addition, when protection function of the stomach wall weakens due to fatigue, malnutrition, anemia, or intake of various medicines, the stomach wall may be directly exposed to attack by stomach acid, resulting in gastritis.

As medicines for gastrointestinal diseases, antacids neutralizing gastric acid already produced without affecting a secretion amount of gastric acid, drugs inhibiting the secretion of gastric acid, secretagogues of prostaglandin, and drugs enhancing defense factors such as a gastric wall coating agent are generally used. Agents used as antacids and secretagogues are prepared using materials artificially produced by chemical methods. Although effects of such artificially produced medicines are not less than those of herbal medicines, the artificially produced medicines exhibit considerable side effects in many cases.

Further, since gastrointestinal diseases often recur even after being cured, it is essential to maintain care, healthy lifestyle, and healthy eating habits after gastrointestinal diseases are cured.

With regard to such gastrointestinal diseases, the health supplement food of the present invention may be taken.

In accordance with a preferred third embodiment of the present invention, a method of preparing a powder formulation for alleviating gastrointestinal disorder symptoms may include the following steps:

S1) a step of cleanly washing black beans, black sesame seeds, corn, adlay, and sorghum, mulberry leaves, mulberry fruits, Polygonum multiflorum Thunberg, dandelion, licorice, kelp, and cactus and then drying the same in the sun until water is completely removed therefrom, followed by completely drying in a warm room for two to three days;

S2) a step of pulverizing each of the materials and preparing a powder; and

S3) a step of mixing the prepared powders such that 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to 15% by weight of dandelion, 1 to 10% by weight of licorice, 2 to 15% by weight of kelp, and to 15% by weight of cactus are included, thereby preparing a powder mixture.

It was confirmed that, when the powder-type health supplement food was taken, gastrointestinal disorder symptoms were alleviated.

Now, the present invention will be described in more detail with reference to the following examples. These examples are provided for illustrative purposes only and should not be construed as limiting the scope and spirit of the present invention and are obvious to those of ordinary skill in the art to which the present invention pertains. In addition, those of ordinary skill in the art may carry out a variety of applications and modifications based on the foregoing teachings within the scope of the present invention, and these modified embodiments may also be within the scope of the present invention.

### Example 1: Pill Preparation

Black beans, black sesame seeds, corn, adlay, sorghum, mulberry leaves, mulberry fruits, dandelion, Polygonum multiflorum Thunberg, licorice, and kelp were washed clean and then were dried in the sun for two days until water is completely removed therefrom, followed by completely drying in a warm room using wood fires for two to three days. Here, mulberry leaves and mulberry fruits were collected when the mulberry fruits were blue, red, and black, and dandelion was collected with roots.

Each of the materials was pulverized, thereby preparing a powder.

800 g (one doe) of each of pulverized black beans, black sesame seeds, corn, adlay, and sorghum was fed into a mixing vessel. Subsequently, 800 g of pulverized mulberry leaves, 800 g of mulberry fruits, 800 g of dandelion, 1200 g (2 geun) of Polygonum multiflorum Thunberg, 300 g (half geun) of licorice, 800 g of kelp, and 800 g of a cactus (Opuntia Humifusa) powder were added thereto, thereby preparing a powder mixture (8.5 kg).

Fermented mulberry fruit solution (500 g) and water (500 g) were added to the powder mixture (8.5 kg) to prepare a pill formulation. Here, as the fermented mulberry fruit solution, a filtrate obtained by mixing mulberry fruits and sugar in a weight ratio of 1:1 and then aging the same for three months, followed by filtration, was used.

### Comparative Example 1

A pill formulation was prepared in the same manner as in Example 1, except that dark purple mulberry fruits were used.

### Experimental Example 1: Hair Loss Alleviation Effect

In this experiment, twenty subjects (40 to 75 years old) who were suffering hair loss were selected and divided into two groups. With water in the morning, lunch and evening for 90 days, Group 1 took 20 pills, which were prepared according to Example 1, and Group 2 took 20 pills, which were prepared according to Example 2. At 1 month, 2 months, and 3 months after ingestion, hair loss alleviation degrees of the subjects were evaluated by a questionnaire to score a hair loss reduction degree, a hair thickness increase degree, and a hair growth promotion degree according to the following criteria. Average values thereof were calculated. Results are summarized in Table 1 below.

5: Very alleviated

4: Alleviated

3: Normal

2: Deteriorated

1: No effect

TABLE 1

| | | After | After | After |
|---|---|---|---|---|
| | | one | two | three |
| | | month | months | months |
| Group 1 | Hair loss alleviation | 4.5 | 4.5 | 4.7 |
| (Example 1) | Hair thickness increase | 4.3 | 4.5 | 4.6 |
| | Hair growth promotion | 4.1 | 4.2 | 4.5 |
| Group 2 | Hair loss alleviation | 3.5 | 3.6 | 3.7 |
| (Comparative | Hair thickness increase | 3.2 | 3.3 | 3.3 |
| Example 1) | Hair growth promotion | 3.4 | 3.5 | 3.6 |

As shown in Table 1, most of the subjects experienced hair loss prevention and hair growth effects. However, in Group 1, superior effects are exhibited. In particular, in the case of Group 1, fluffy hairs begin to grow in a hairless area from one month after intake, and, after three months, fluffy hairs are observed from all subjects.

In addition, it was confirmed that hair thickness also increased. In the case of a 75-year-old female, white hair was changed to black hair. In the case of a 45-year-old male, hair growth was observed from one week after intake and, after three months, alopecia areata was almost cured. Here, no adverse events were observed in all the patients who participated in the clinical trial.

### Experimental Example 2: Blood Glucose Regulation

Sixteen adults having a fasting blood glucose measurement value of 126 or more, which was measured at a health checkup institution in the last 3 months, were selected and divided into two groups. With water in the morning, lunch and evening for 90 days, Group 1 took 20 pills, which were prepared according to Example 1, and Group 2 took 20 pills, which were prepared according to Example 2.

In addition, an individual self-monitoring blood glucose meter was given to each subject such that a fasting blood glucose level was measured before first taking the pills and, after 30 days, a fasting blood glucose level was measured again.

In Table 2 below, initial blood glucose levels and blood glucose levels at one month, two months, and three months after intake of the respective experimental groups are shown.

TABLE 2

| | | After | After | After |
|---|---|---|---|---|
| | | one | two | three |
| Blood glucose (mg/dL) | | month | months | months |
| Group 1 | Average of initial | 135 | 127 | 115 |
| (Example 2) | blood glucose levels | | | |
| | Average of final | 127 | 115 | 106 |
| | blood glucose levels | | | |
| | Difference between | 8 | 8 | 9 |
| | initial blood glucose | | | |
| | level average and | | | |
| | final blood glucose | | | |
| | level average | | | |
| Group 2 | Average of initial | 138 | 133 | 125 |
| (Comparative | blood glucose levels | | | |
| Example 2) | Average of final | 137 | 125 | 121 |
| | blood glucose levels | | | |
| | Difference between | 1 | 8 | 4 |
| | initial blood glucose | | | |
| | level average and | | | |
| | final blood glucose | | | |
| | level average | | | |

Referring to Table 2, it can be confirmed that, when the pill formulation prepared according to Example 1 is taken, a blood glucose level is further decreased, compared to the case in which the pill formulation according to Comparative Example 2 is taken.

### Experimental Example 3: Alleviation and Relief Effect on Gastric Disorder Symptoms

In this experiment, twenty subjects (40 to 75 years old) having a chronic gastrointestinal disease were selected and divided into two groups. With water in the morning, lunch and evening for 90 days, Group 1 took 20 pills, which were prepared according to Example 1, and Group 2 took 20 pills, which were prepared according to Example 2. At 1 month, 2 months, and 3 months after the ingestion, heartburn, abdominal bloating and nausea of the subjects were scored according to the following criteria. Average values thereof were calculated. Results are summarized in Table 3 below.

5: Very alleviated

4: Alleviated

3: Normal

2: Deteriorated

1: No effect

TABLE 3

| | | After | After | After |
|---|---|---|---|---|
| | | one | two | three |
| | | month | months | months |
| Group 1 | Heartburn | 4.2 | 4.3 | 4.8 |
| (Example 2) | Abdominal | 4.3 | 4.5 | 4.6 |
| | bloating | | | |
| | Nausea | 4.5 | 4.6 | 4.9 |
| Group 2 | Heartburn | 3.5. | 3.4 | 3.2 |
| (Comparative | Abdominal | 3.4 | 3.5 | 3.6 |
| Example 2) | bloating | | | |
| | Nausea | 3.6 | 3.5 | 3.7 |

Referring to Table 3, it can be confirmed that, in most of the subjects, the symptoms observed in a chronic gastrointestinal disease are alleviated. In addition, it can be confirmed that, when the pill formulation prepared according to Example 1 is taken, superior effect is exhibited, compared to the case in which the pill formulation according to Comparative Example 2 is taken.

### INDUSTRIAL APPLICABILITY

A health supplement food according to the present invention may be mass-produced and marketed for public health promotion.

1. A health supplement food, wherein the health supplement food is prepared by mixing dried powders, wherein the dried powders include 5 to 15% by weight of black beans, 5 to 15% by weight of black sesame seeds, 5 to 15% by weight of corn, 5 to 15% by weight of adlay, 5 to 15% by weight of sorghum, 5 to 15% by weight of mulberry leaves, 5 to 15% by weight of mulberry fruits, 7 to 20% by weight of Polygonum multiflorum Thunberg, 5 to 15% by weight of dandelion, 1 to 10% by weight of licorice, 2 to 15% by weight of kelp, and 5 to 15% by weight of cactus based on solid content.

2. The health supplement food according to claim 1, wherein the mulberry leaves and the mulberry fruits are simultaneously harvested from Morus bombycis, wherein the mulberry fruits are harvested when blue, red, and dark purple mulberry fruits are mixed.

3. The health supplement food according to claim 1, wherein the health supplement food has a pill, granule, tablet, powder, capsule, or beverage formulation.
